(19) United States
(12) Patent Application Publication
(10) Pub. No.: US 2016/0278786 A1
(43) Pub. Date: Sep. 29, 2016

(54) BONE WAX DISPENSER

(71) Applicant: Resorbable Orthopedic Products, LLC, Addison, TX (US)

(72) Inventor: Barry E. Constantine, Island Heights, NJ (US)

(21) Appl. No.: 15/080,008

(22) Filed: Mar. 24, 2016

Related U.S. Application Data
(60) Provisional application No. 62/137,746, filed on Mar. 24, 2015.

(51) Int. Cl. A61B 17/12

(52) U.S. Cl. CPC A61B 17/12186; A61B 17/12195; A61B 2017/1205; A61B 2017/12004; A61B 2017/00526

(57) ABSTRACT

A dispenser comprising a tubular housing, an adjustable base configured to move from one end of the tubular housing to a second end of the tubular housing and bone wax disposed within the tubular housing.

### RELATED APPLICATION

The present application claims priority to U.S. Provisional Patent Application No. 62/137,746, filed Mar. 24, 2015, which is hereby incorporated by reference for all purposes as if set forth herein in its entirety.

### TECHNICAL FIELD

The present disclosure relates generally to medical materials, and more specifically to a dispenser for bone wax and other materials and a method of use.

### BACKGROUND OF THE INVENTION

Medical materials are often packaged in a pouch or other software structures, and are difficult to use when deployed from such packaging.

### SUMMARY OF THE INVENTION

A dispenser comprising a tubular housing, an adjustable base configured to move from one end of the tubular housing to a second end of the tubular housing and bone wax disposed within the tubular housing, which functions as a barrier between the water soluble wax and a wet surgical glove to improve the handling of the water soluble wax.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE SEVERAL VIEWS OF THE DRAWINGS

Aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views, and in which:

FIG. 1 is a diagram of a dispenser for bone wax, in accordance with an exemplary embodiment of the present disclosure;

FIG. 2 is a diagram of a device for dispensing bone wax, in accordance with an exemplary embodiment of the present disclosure;

FIG. 3 is a diagram of a device for dispensing bone wax, in accordance with an exemplary embodiment of the present disclosure; and

FIG. 4 is a diagram of a device for dispensing bone wax, in accordance with an exemplary embodiment of the present disclosure.

### DETAILED DESCRIPTION OF THE INVENTION

In the description that follows, like parts are marked throughout the specification and drawings with the same reference numerals. The drawing figures might not be to scale and certain components can be shown in generalized or schematic form and identified by commercial designations in the interest of clarity and conciseness.

Water soluble bone waxes address the bone wax granuloma issue associated with non-resorbable bone wax, a sterile blend of water soluble alkylene oxide copolymers such as HemaQuell™, which is a commercially available product from Wound Management Technologies, or other suitable materials. Such water soluble bone waxes becomes slippery when manipulated with a wet surgical glove into a bony defect, such that control and placement is compromised. The use of the tube delivery system as disclosed herein improves the handling characteristics of a water soluble wax during a surgical procedure, which helps to ensure proper positioning and placement of the product to control bleeding.

A water soluble surgical implant bone wax material can be used for the control of bleeding from bone surfaces by way of tamponade, providing a physical, resorbable, barrier to blood flow. Such bone wax materials should be warmed slightly to facilitate the extrusion of the hemostatic material from the product delivery tube package, which complicates the delivery of the material. As such, any delivery mechanism for a bone wax material should be configured for immersion in warm sterile water, or in other suitable manners. In addition, the bone wax material needs to be sterilized, such as by gamma irradiation or in other suitable manners, such that any delivery mechanism must be capable of supporting such sterilization procedures and not impair or prevent sterilization.

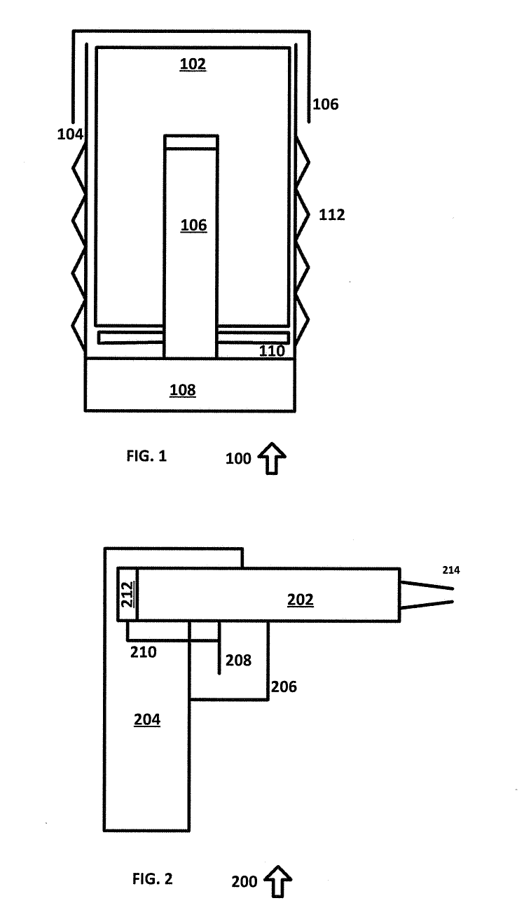

FIG. 1 is a diagram of a dispenser 100 for bone wax, in accordance with an exemplary embodiment of the present disclosure. Dispenser 100 includes bone wax 102, which can be Signafuse Bioactive Bone Graft Putty available from Resorbable Orthopedic Products, LLC or Fort Worth, Tex., or other suitable materials. Bone wax 102 is disposed within cylinder 104, which can be formed from plastic, rubber, polymers, metal, glass or other suitable materials. If the cylinder 104 is formed from any material that shields the bone wax 102 from gamma radiation or other sterilization mechanism, then an alternative form of sterilization or sterilization procedures will need to be applied, such that a non-metallic material is more suitable to gamma radiation sterilization. Cylinder 104 includes center post 106, base 108 and platform 110, which are configured to cause platform 110 to move along post 106 in a first direction when base 108 is rotated in a clockwise direction, and to move along post 106 in a second direction when base 108 is rotated in a counter-clockwise direction. For example, post 106 and the inside of cylinder 104 can include threads or other suitable circumferential channels that cause platform 110 to move outward along post 106 from base 108 to the far end of post 106 when base 108 is rotated in a clockwise direction, and to move backwards towards base 108 when base 108 is rotated in a counter-clockwise direction. Likewise, other suitable motive mechanisms can also or alternatively be used.

Cylinder 104 can also include an external texture 112, such as a ribbed surface or other suitable coverings, to improve the ability of a surgeon or other medical services provider to hold on to cylinder 104 with a wet surgical glove. Because accurate placement of the water soluble wax is important, the ability to precisely deliver a controllable amount of water soluble bone wax is important during a surgical operation, when a surgeon or other medical services provider will be wearing latex surgical gloves. As such, external texturing 112 can be used to improve the ability to grip cylinder 104 in such situations. Likewise, base 108 can include other suitable devices to improve the ability to provide a predetermined amount of water soluble bone wax under surgical conditions, such as a spring-loaded lever assembly that causes a predetermined amount of water soluble bone wax to be provided, a plunger assembly with suitable markings, an electronic control or other suitable devices.

Cap 106 is disposed at the end of cylinder 104, and maintains bone wax 102 in a sterile environment prior to use. Because bone wax 102 is used for medical purposes, maintaining a sterile environment during manufacturing, shipment and storage is a primary concern. As such, cap 106 can include threads, locking devices or other structures that secure cap 106 to cylinder 104 after it is installed during the manufacturing process, and which form a visible break after opening, to prevent dispenser 100 from being used more than once. Cap 106 can form a water-tight seal, to allow dispenser 100 to be immersed in warm sterile water for heating prior to use, or to otherwise be warmed prior to use to improve the ease of application. Cylinder 104 can also be formed so as to facilitate heating in such manners, such as by restricting the wall thickness of the material used to form cylinder 104 to 1 millimeter or less, so as to reduce any impediment to heat transfer. Likewise, struts or other mechanical supports (such as in place of external texture 112) can be provided to improve the mechanical strength of cylinder 104 to allow the thickness of the material to be reduced to 0.25 millimeter or less without loss of mechanical integrity.

Dispenser 100 can be fabricated in the following process. First, the separate component parts of dispenser 100 can be sterilized in a suitable sterilizer device, such as an autoclave, using ultraviolet or microwave radiation, using alcohol or in other suitable manners. The component parts can then be assembled using sterile equipment, and a protective layer of isolating material can be disposed along the inner surface of cylinder 104, in addition to sterilizing substances such as iodine compounds, chlorine compounds or other suitable compounds. Heated liquid bone wax 102 can then be poured into cylinder 104 and allowed to cool. In addition, other suitable sealing compounds can be used to ensure that bone wax 102 does not penetrate into the assembly of center post 106, base 108 and platform 110, or to otherwise prevent fouling. After cooling in a sterile environment, end cap 106 can be installed, an additional heat wrap layer or other suitable sterile package can be disposed around dispenser 100, or other suitable processes and materials can also or alternatively be used.

In use, dispenser 100 can be heated to a suitable temperature, such as between 100 to 140 degrees Fahrenheit, to improve the ease of operation for dispensing bone wax 102 from dispenser 100. The user then rotates base 108 in the appropriate direction to cause bone wax 102 to be dispensed in a suitable amount and to a suitable location. In one exemplary embodiment, dispenser 102 can include a locking mechanism to prevent bone wax 102 from being retracted back into dispenser 100, such as to avoid potential contamination of sterile bone wax 102 within dispenser 100.

FIG. 2 is a diagram of a device 200 for dispensing bone wax, in accordance with an exemplary embodiment of the present disclosure. Device 200 includes tube 202, which has a bone wax substance disposed within as previously disclosed. Grip 204 is coupled to tube 202 and provides a handle by which to hold device 200. Grip 204 can include ribs, texture, a roughened surface or other suitable surface treatments or coverings to improve the ability of device 200 to be held by a surgeon or other suitable medical personnel during a medical procedure. Finger guard 206 and trigger assembly 208 allow the user to control an amount of bone wax that is to be provided, in conjunction with transfer assembly 210 and actuator 212. In one exemplary embodiment, transfer assembly 210 can be configured to cause actuator 212 to rotate by a predetermined amount whenever trigger assembly 208 is depressed by a user, such as by using a spring-loaded lever arm that rotates actuator 212 and then returns to a reset position or in other suitable manners. In this manner, a user can dispense a predetermined amount of bone wax from cylinder 202 by pressing trigger assembly 208.

Depending upon the consistency of the bone wax, tube 202 can also or alternatively have an applicator tip 214 that can be trimmed to increase an amount of bone wax that is provided to a location, such as if the consistency of the bone wax is too viscous to be dispensed accurately without an additional supporting delivery structure.

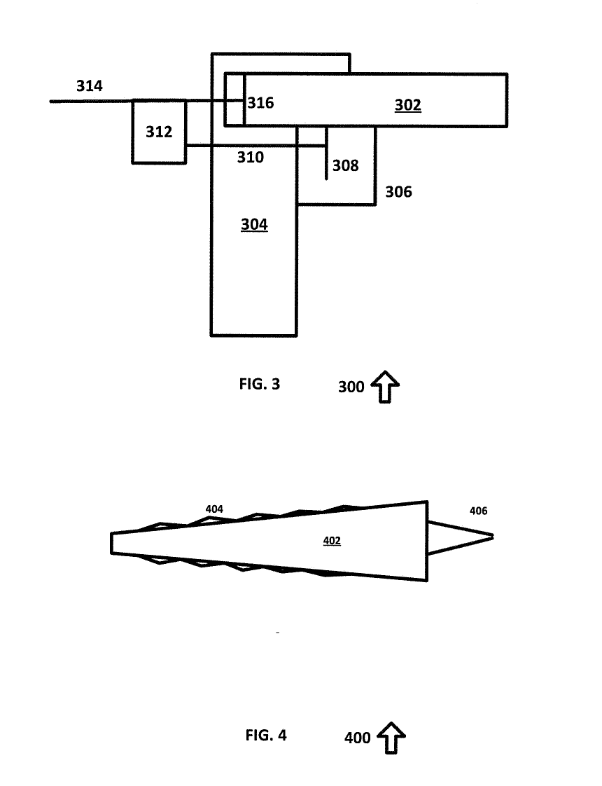

FIG. 3 is a diagram of a device 300 for dispensing bone wax, in accordance with an exemplary embodiment of the present disclosure. Device 300 includes tube 302, which has a bone wax substance disposed within as previously disclosed. Grip 304 is coupled to tube 302 and provides a handle by which to hold device 300. Grip 304 can include ribs, texture, a roughened surface or other suitable surface treatments or coverings to improve the ability of device 300 to be held by a surgeon or other suitable medical personnel during a medical procedure. Finger guard 306 and trigger assembly 308 allow the user to control an amount of bone wax that is to be provided, in conjunction with transfer assembly 310 and plunger controller 312. In one exemplary embodiment, transfer assembly 310 can be configured to cause plunger controller 312 to move plunger 314 inwards by a predetermined amount whenever trigger assembly 308 is depressed by a user, such as by using a spring-loaded lever arm that moves plunger 314 and then returns to a reset position or in other suitable manners. In this manner, a user can dispense a predetermined amount of bone wax from cylinder 302 by pressing trigger assembly 308 and causing plunger 314 to move base 316 by a predetermined amount. Likewise, device 300 can be used without a trigger assembly 308 or grip 304, and can instead utilize a textured surface on tube 302 and plunger 314, which can be manually depressed by the user. Although this embodiment may require two hands to operate device 300, it allows device 300 to be smaller and mechanically simpler.

FIG. 4 is a diagram of a device 400 for dispensing bone wax, in accordance with an exemplary embodiment of the present disclosure. Device 400 includes tube 402 of bone wax material, with a flexible textured surface 404 and an adjustable dispenser tip 406. In this exemplary embodiment, the flexible textured surface 404 of tune 402 allows a user-controllable amount of bone wax to be dispensed through dispenser tip 406. Dispenser tip 406 can be trimmed by the user to increase the amount of bone wax that is provided to a location. In addition, the viscosity of the bone wax can be changed to optimize the bone wax for delivery through device 400 or other devices as disclosed herein, such as by increasing or decreasing an amount of water in the bone wax, by increasing or decreasing an amount of beeswax in the bone wax, by increasing or decreasing an amount of isopropyl palmitate in the bone wax, by heating or cooling the bone wax to a predetermined temperature, or in other suitable manners.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

What is claimed is:

1. A dispenser comprising:
a tubular housing;
an adjustable base configured to move from one end of the tubular housing to a second end of the tubular housing; and
bone wax disposed within the tubular housing.

2. The dispenser of claim 1 further comprising a cap disposed on the tubular housing opposite the adjustable base.

3. The dispenser of claim 2 further comprising a water-tight seal formed between the cap and the tubular housing.

4. The dispenser of claim 1 wherein the tubular housing has a thickness of less than one millimeter to facilitate heat transfer to the bone wax.

5. The dispenser of claim 1 wherein the tubular housing has a thickness of less than one quarter millimeter to facilitate heat transfer to the bone wax.

6. The dispenser of claim 1 further comprising an external texture disposed on the tubular housing.

7. The dispenser of claim 1 wherein the adjustable base further comprises a plunger mechanism for controlling delivery of the bone wax.

8. The dispenser of claim 1 wherein the adjustable base further comprises:
a plunger mechanism for controlling delivery of the bone wax; and
a trigger mechanism coupled to the plunger mechanism and configured to controllably adjust a position of the plunger mechanism.

9. A method for fabricating a dispenser, comprising:
sterilizing a tubular housing and an adjustable base configured to move from one end of the tubular housing to a second end of the tubular housing;
sterilizing bone wax that is configured for placement within the tubular housing; and
disposing the sterilized bone wax within the sterilized tubular housing.

10. The method of claim 9 further comprising disposing a sterilized cap on the tubular housing opposite the adjustable base.

11. The method of claim 10 further comprising forming a sterilized water-tight seal between the sterilized cap and the sterilized tubular housing.

12. A method of fabricating a dispenser having a tubular housing, an adjustable base configured to move from one end of the tubular housing to a second end of the tubular housing, bone wax disposed within the tubular housing, a cap disposed on the tubular housing opposite the adjustable base, a water-tight seal formed between the cap and the tubular housing, wherein the tubular housing has a thickness of less than one millimeter to facilitate heat transfer to the bone wax, an external texture disposed on the tubular housing, a plunger mechanism for controlling delivery of the bone wax and a trigger mechanism coupled to the plunger mechanism and configured to controllably adjust a position of the plunger mechanism, the method comprising:
sterilizing a tubular housing and an adjustable base configured to move from one end of the tubular housing to a second end of the tubular housing;
sterilizing bone wax that is configured for placement within the tubular housing;
disposing the sterilized bone wax within the sterilized tubular housing;
disposing a sterilized cap on the tubular housing opposite the adjustable base; and
forming a sterilized water-tight seal between the sterilized cap and the sterilized tubular housing.
